(19) United States
(12) Patent Application Publication
(10) Pub. No.: US 2013/0303955 A1
(43) Pub. Date: Nov. 14, 2013

(54) OFF-THE-SHELF ADJUSTABLE BRACE FOR TREATING SCOLIOSIS AND ASSOCIATED METHODS

(71) Applicant: Wallace C. BEITL, Orlando, FL (US)

(72) Inventor: Wallace C. BEITL, Orlando, FL (US)

(21) Appl. No.: 13/469,264

(22) Filed: May 11, 2012

(51) Int. Cl. A61F 5/02

(52) U.S. Cl. 602/19

(57) ABSTRACT

The adjustable scoliosis brace is for treating a spinal curve in a patient and includes front and back vertical support pillars each having a longitudinal connection plate including upper and lower variable connection portions, and a rotatable arm coupled to the upper variable connection portions. A girdle device is coupled to the front and back vertical support pillars. The girdle device includes waist engaging members contoured to inhibit movement of the brace on a waist of the patient, adjustable coupling features configured to couple the plurality of waist engaging members together, and variable connection features configured to couple the plurality of waist engaging members to the longitudinal connection plates. An upper thoracic pad is configured to be coupled between the rotatable arms. A lower thoracic pad is configured to be coupled between the longitudinal connection plates of the front and back vertical support pillars.

### FIELD OF THE INVENTION

The present invention relates to the field of orthotics, and, more particularly, to orthotic braces to treat scoliosis and related methods.

### BACKGROUND OF THE INVENTION

Scoliosis, which is an abnormal curvature of the spine coupled with vertebral rotation, is most commonly found in adolescent females. This abnormal curvature and rotation causes deformity of the rib cage which results in asymmetries of the trunk. In previous studies, seven features of scoliosis were identified: shoulder height and shoulder angle differences, pelvis asymmetry, decompensation, waist crease, scapula height difference and waist asymmetry. These identified features account for 85% of the overall impression of trunk deformity.

Clinicians have few non-surgical treatment tools for children with potentially progressive spinal deformities such as scoliosis. Brace treatment is most commonly used despite poor compliance and uncertainty as to effectiveness. The Boston and Charleston braces are most frequently prescribed due to their low profile. To be effective, the Boston brace is required to be worn for up to 23 hours/day. The nighttime Charleston brace must be worn 8 hours/night.

Currently, bracing is the only non-operative treatment for adolescent idiopathic scoliosis advocated by the spine medical community. All other forms of non-surgical treatment for adolescent idiopathic scoliosis, including chiropractic or osteopathic manipulation, exercise or other manual treatments, nutrition, acupuncture, etc., have not been proven to be effective in preventing progression or reducing the curvature.

Patients with a curve that is between 20 to 30 degrees will usually be observed at 4 to 6 month intervals with an x-ray test to measure the curve. Any progression that is less than 5 degrees is not considered significant. If the curve progresses more than 5 degrees, then the curve will need treatment. In general, treatment with a brace will be recommended for patients with a curve that measures 25 to 40 degrees, and substantial growth remaining (patients who are skeletally immature).

The primary goal of orthotic treatment (a back brace) for idiopathic scoliosis is to stop the progression of the spinal curve. Bracing does not typically reduce the degree of the curve/amount of angulation already present. Since bracing may only work to stop the progression of the curvature in a growing child, it is not typically used for those children who are already skeletally mature or almost mature. It is mainly used for younger children (e.g. girls who are about 11 to 13 years old, and boys who are about 12 to 14 years old).

Brace treatment is usually used in girls up to one year after menarche, the onset of the female menstrual cycle. If an older child has a curve greater than 30 degrees and is almost mature, his or her curvature will typically be treated with observation only, as there is little growth left and bracing will be unlikely to be as effective. Curves that tend to continue to progress after skeletal maturity are those that are greater than 50 degrees in angulation, so the treatment objective with a back brace is to try to get the child into adulthood with less than a 50 degree curvature.

While it is non-invasive, bracing for scoliosis may be nonetheless a very difficult treatment option for many patients. Adolescence is usually difficult enough without having to appear different than one's peers by wearing a brace. Compliance with wearing a brace is very important, as curves that progress can require fusion surgery and lead to potential long-term health problems.

There are a number of bracing options, and the physician may recommend a particular back brace and bracing schedule based on factors, such as, the location of the child's curve and degree of curvature. Compliance with wearing the back brace as prescribed is important to the success of bracing treatment.

There are several types of commonly used scoliosis braces. The most common form of a Thoraco-Lumbo-Sacral-Orthosis (TLSO) brace is called the “Boston brace”, and it may be referred to as an “underarm” brace. This brace is fitted to the child's body and custom molded from plastic. It works by applying three-point pressure to the curvature to prevent its progression.

It can be worn under clothing and is typically not noticeable. The TLSO brace is usually worn 23 hours a day, and it can be taken off to swim, play sports or participate in gym class during the day. This type of brace is usually prescribed for curves in the lumbar or thoraco-lumbar part of the spine.

A Cervico-Thoraco-Lumbo-Sacral-Orthosis (known as a Milwaukee brace) is similar to the TLSO described above, but also includes a neck ring held in place by vertical bars attached to the body of the brace. It is usually worn 23 hours a day, and can be taken off to swim, play sports or participate in gym class during the day. This type of brace is often prescribed for curves in the thoracic spine.

A Charleston Bending Brace is also called a “nighttime” brace because it is only worn while sleeping. A Charleston brace is molded to the patient while they are bent to the side, and thus applies pressure and bends the child against the curve. This pressure imparts the corrective action of the brace. Patients can go to school and participate in sports normally without their friends even knowing they have scoliosis and wear a brace, avoiding any potential negative stigma. For example, U.S. Pat. No. 4,688,558 to Hooper Jr. et al. describes such a brace.

Many studies have shown that the Charleston nighttime brace is as effective as the above-described 23-hour-a-day brace. Curves in the 20 to 40 degree range, and the apex of the curve below the level of the shoulder blade are desirable for the Charleston brace to be effective.

Other braces are disclosed in U.S. Pat. No. 7,967,767 to Ogilvie, U.S. Pat. No. 6,676,617 to Miller and U.S. Pat. No. 4,285,336.

The typical brace is custom molded and not expandable. There is a need for an “off-the-shelf” brace that is effective in treating a spinal curve and can be expanded and adjusted as needed by the doctor for the patient.

### SUMMARY OF THE INVENTION

In view of the foregoing background, it is therefore an object of the present invention to provide an effective treatment for a spinal curve including use of an “off-the-shelf” brace that can be expanded and adjusted as needed.

This and other objects, features, and advantages in accordance with the present invention are provided by an adjustable scoliosis brace for treating a spinal curve in a patient and including front and back vertical support pillars each comprising a longitudinal connection plate including upper and lower variable connection portions, and a rotatable arm coupled to the upper variable connection portion of the longitudinal connection plate and extending upwardly therefrom. A girdle device is coupled to the front and back vertical support pillars and configured to surround a waist of the patient. The girdle device includes a plurality of waist engaging members contoured to inhibit movement of the brace on a waist of the patient, adjustable coupling features provided on an external surface of the plurality of waist engaging members and configured to couple the plurality of waist engaging members together, and variable connection features configured to couple the plurality of waist engaging members to the lower variable connection portions of the longitudinal connection plates. An upper thoracic pad is configured to be coupled between the rotatable arms of the front and back vertical support pillars and positioned under one shoulder of the patient. A lower thoracic pad is configured to be coupled between the longitudinal connection plates of the front and back vertical support pillars and positioned between the girdle device and the other shoulder of the patient.

Padding may be positioned on interior surfaces of the front and back vertical support pillars, the girdle device and the upper and lower thoracic pads. The variable connection features of the girdle device may include horizontal slots adjacent ends of the plurality of waist engaging members, and the lower variable connection portions of the longitudinal connection plates may include vertical slots. Fasteners may be configured to fasten ends of the plurality of waist engaging members to the lower variable connection portions of the longitudinal connection plates via the horizontal and vertical slots.

The upper variable connection portion of the longitudinal connection plate may include an arcuate slot or a plurality of setting holes. As such, the rotatable arm may be coupled to the upper variable connection portion via a first fastener at an axis of rotation, and a second fastener extending through the arcuate slot or through at least one of the setting holes.

The adjustable coupling features provided on the external surface of the plurality of waist engaging members may comprise straps and associated buckles. Also, the front and back vertical support pillars may comprise plastic and/or metal front and back vertical support pillars. The plurality of waist engaging members may comprise plastic waist engaging members.

The upper thoracic pad may be coupled between the rotatable arms of the front and back vertical support pillars via at least one strap and associated buckle. Likewise, the lower thoracic pad may be coupled between the longitudinal connection plates of the front and back vertical support pillars via at least one strap and associated buckle.

A method aspect is directed to treating a spinal curve in a patient with an adjustable scoliosis brace, the method including providing front and back vertical support pillars each comprising a longitudinal connection plate including upper and lower variable connection portions, and a rotatable arm coupled to the upper variable connection portion of the longitudinal connection plate and extending upwardly therefrom. The method includes coupling a girdle device to the front and back vertical support pillars and positioning the girdle device to surround a waist of the patient.

The girdle device includes a plurality of waist engaging members contoured to inhibit movement of the brace on a waist of the patient, adjustable coupling features provided on an external surface of the plurality of waist engaging members and configured to couple the plurality of waist engaging members together, and variable connection features configured to couple the plurality of waist engaging members to the lower variable connection portions of the longitudinal connection plates.

The method further includes coupling an upper thoracic pad between the rotatable arms of the front and back vertical support pillars and positioning under one shoulder of the patient, and coupling a lower thoracic pad between the longitudinal connection plates of the front and back vertical support pillars and positioning between the girdle device and the other shoulder of the patient.

### BRIEF DESCRIPTION OF THE DRAWINGS

FIG. 1 is front view of an adjustable scoliosis brace according to a present embodiment.

FIG. 2 is back view of the adjustable scoliosis brace of FIG. 1.

FIG. 3 is a perspective view of the adjustable scoliosis brace of FIGS. 1 and 2.

FIGS. 4A-4D are various perspective views of an embodiment of the adjustable scoliosis brace positioned on a patient.

FIG. 5 is a front elevational view of a front portion of an adjustable scoliosis brace according to another embodiment.

FIG. 6 is a rear elevational view of the front portion of the adjustable scoliosis brace of FIG. 5.

FIGS. 7A and 7B are perspective views of longitudinal connection plate embodiments for the present adjustable scoliosis brace.

FIGS. 8A and 8B are perspective views of rotatable arm embodiments for the present adjustable scoliosis brace.

FIG. 9 is a rear view of another embodiment of an adjustable scoliosis brace for treating a double curve.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

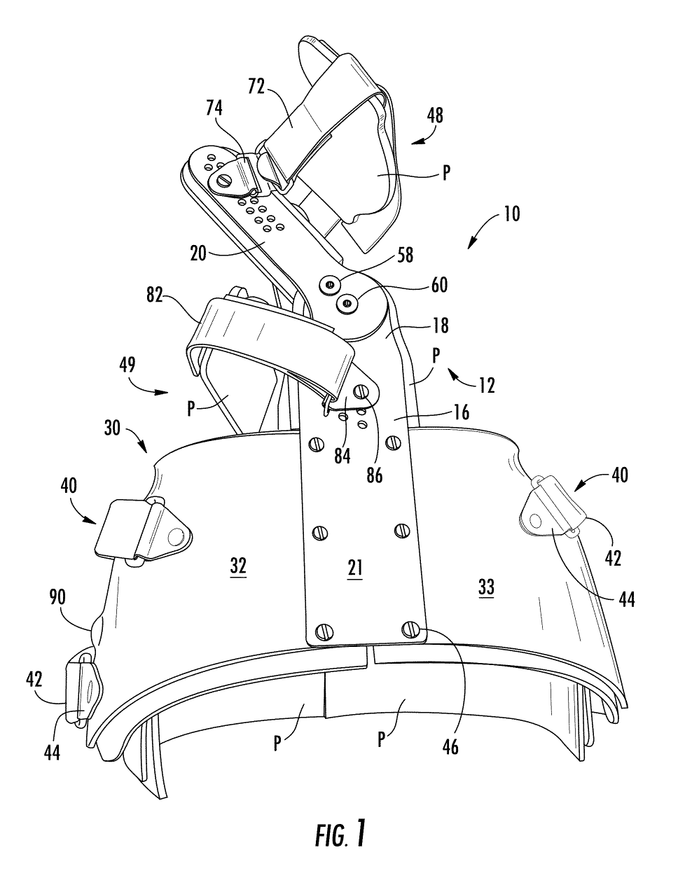

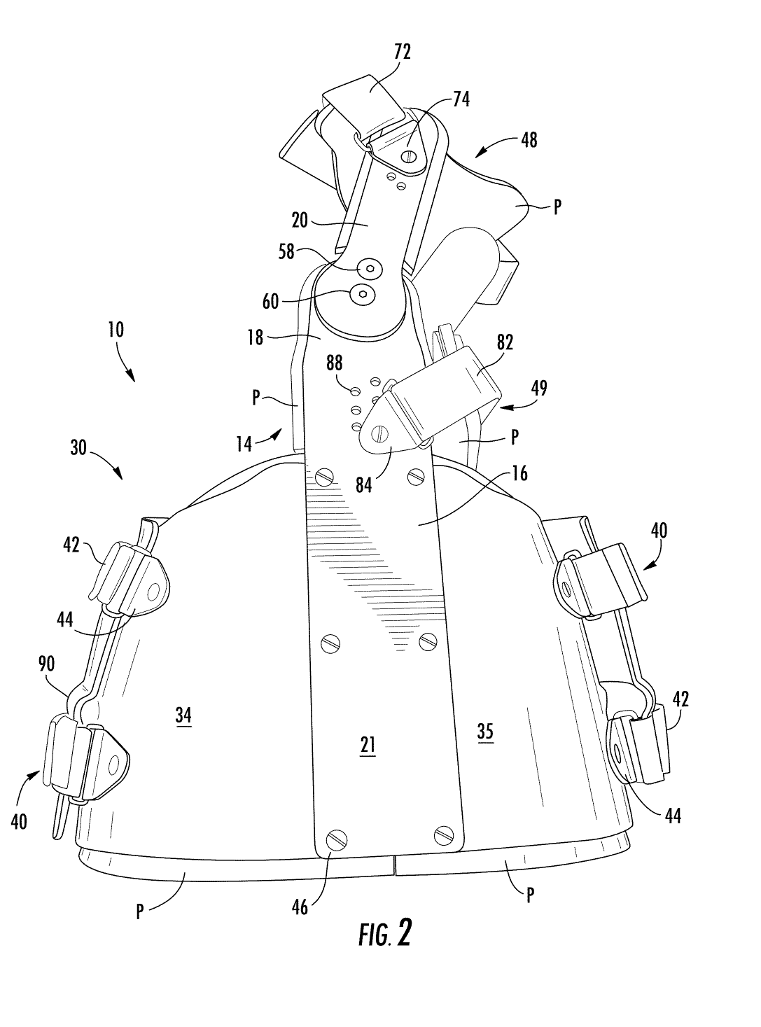

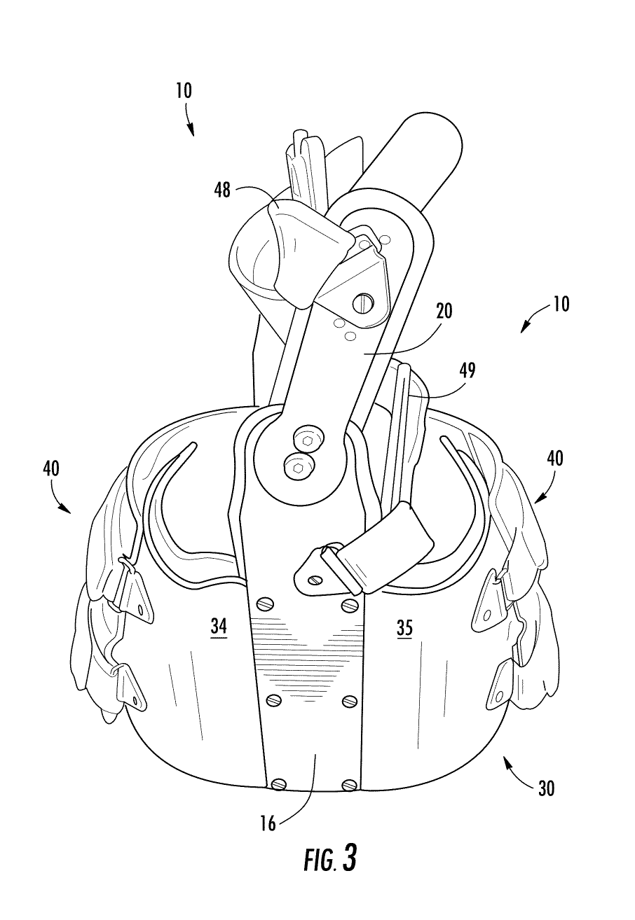

Referring initially to FIGS. 1-4, an expandable, adjustable, off-the-shelf brace 10 that is effective in treating a spinal curve will be described. As with the Charleston Bending Brace discussed above, the present brace 10 may also be referred to as a “nighttime brace” because it may only worn while sleeping. Patients can go to school and participate in sports normally without their friends even knowing they have scoliosis and wear a brace, avoiding any potential negative stigma. However, unlike the conventional Charleston Bending Brace, the present brace 10 is expandable and adjustable, and does not need to be custom molded to the patient's body. Moreover, the user does not need to obtain an entirely new brace for different stages of treatment as the various features of the brace 10 are configured to be updated or upgraded as needed.

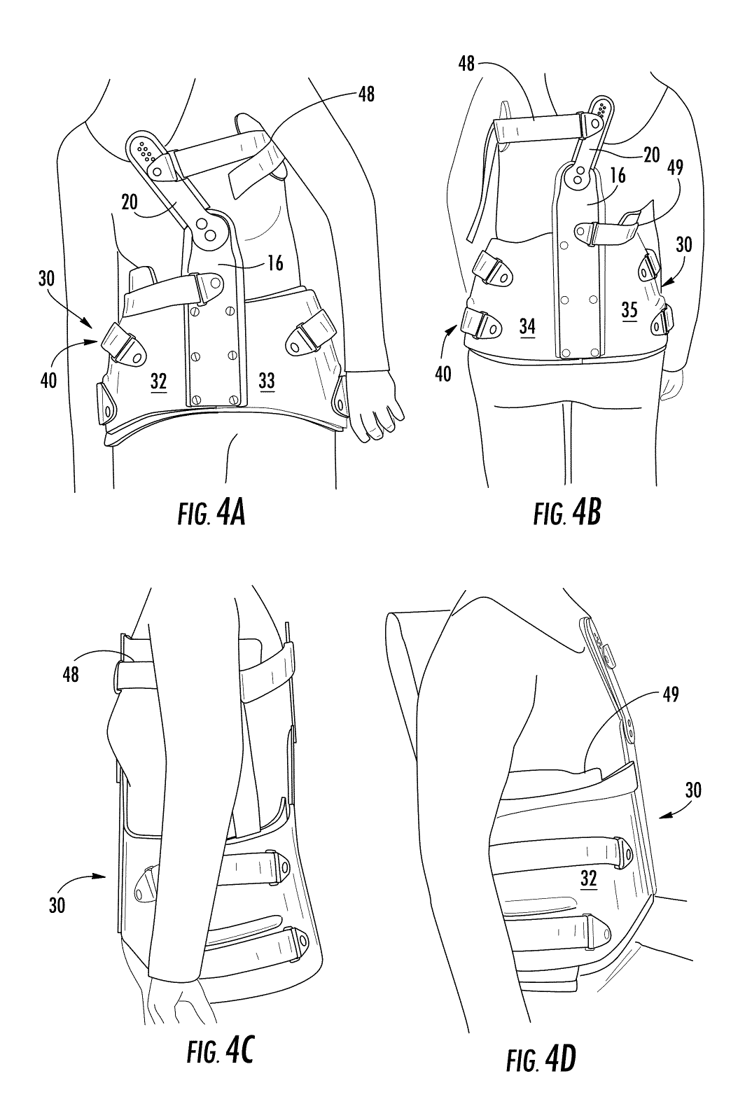

The adjustable scoliosis brace 10 includes front and back vertical support pillars 12/14 each comprising a longitudinal connection plate 16 including upper and lower variable connection portions 18/21, and a rotatable arm 20 coupled to the upper variable connection portion of the longitudinal connection plate and extending upwardly therefrom. A girdle device 30 is coupled to the front and back vertical support pillars 12/14 and configured to surround a waist of the patient (e.g. as illustrated in FIGS. 4A-4D). The girdle device 30 includes a plurality of waist engaging members 32-35 contoured to inhibit movement of the brace on a waist of the patient. Adjustable coupling features 40, such as straps 42 and buckles 44, are provided on an external surface of the plurality of waist engaging members and configured to couple the plurality of waist engaging members together. Any number of straps 42 and buckles 44 may be provided to ensure coupling of the waist engaging members 32-35. Variable connection features 46 are configured to couple the plurality of waist engaging members 32-35 to the lower variable connection portions 20 of the longitudinal connection plates 16.

An alignment feature 90, such as a ridge or contour, for example, may be provided on the waist engaging members 32-35 to prevent such members from sliding relative to one another.

An upper thoracic pad 48 is configured to be coupled between the rotatable arms 20 of the front and back vertical support pillars 12, 14 and positioned under one shoulder of the patient (e.g. as illustrated in FIGS. 4A-4C). A lower thoracic pad 49 is configured to be coupled between the longitudinal connection plates 16 of the front and back vertical support pillars 12, 14 and positioned between the girdle device 30 and the other shoulder of the patient (e.g. as illustrated in FIGS. 4A, 4B and 4D).

The upper thoracic pad 48 is adjusted or otherwise set at a desired angle via the rotatable arm 20 and then secured or locked into position via fasteners 58, 60 as is discussed below. The lower thoracic pad 49 is adjusted or otherwise set at a desired position via the positioning holes 88, for example, as will be discussed in further detail below.

Padding P is illustratively positioned on interior surfaces of the front and back vertical support pillars 12, 14, the girdle device 30 and the upper and lower thoracic pads 48, 49. The padding may be attached to the interior surfaces via adhesive or hook and loop fasteners, for example.

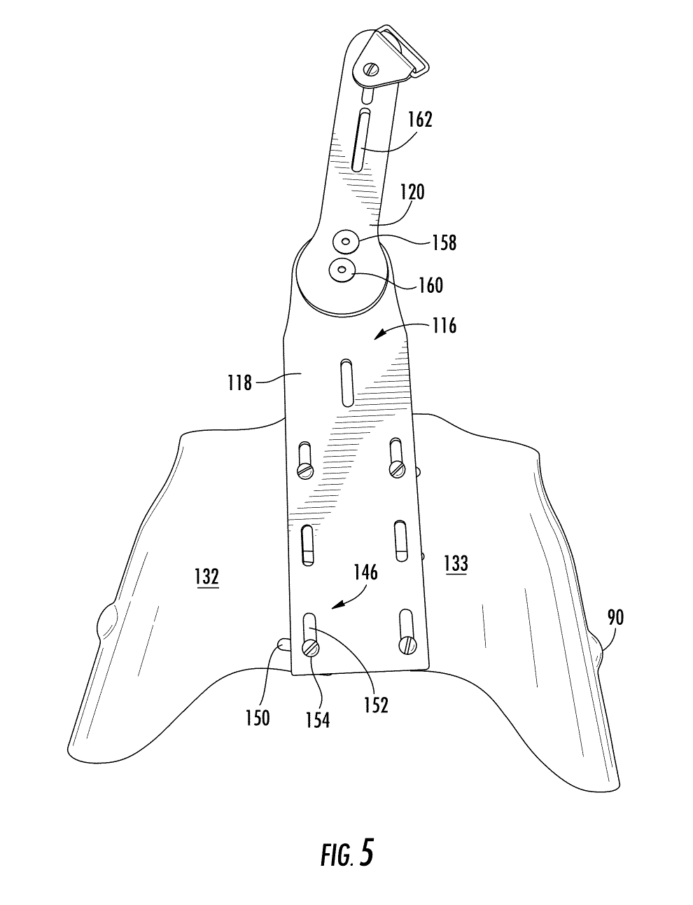

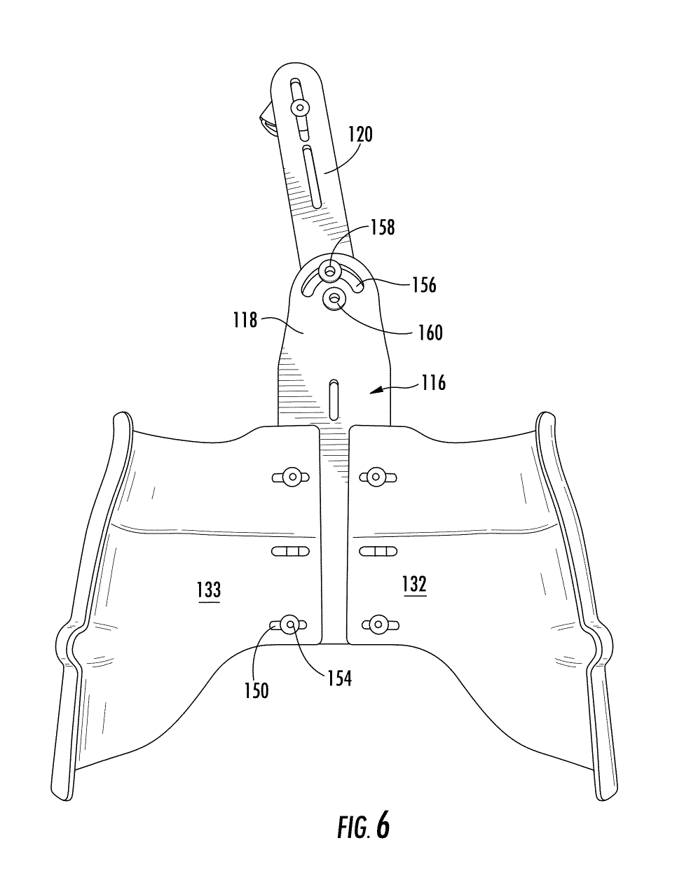

Referring additionally to the embodiment illustrated in FIGS. 5 and 6, the variable connection features 146 of the girdle device 30 may include horizontal slots 150 adjacent ends of the plurality of waist engaging members 132, 133. The lower variable connection portions 121 of the longitudinal connection plates 116 may include vertical slots 152. Fasteners 154 may be configured to fasten ends of the plurality of waist engaging members 132, 133 to the lower variable connection portions 121 of the longitudinal connection plates 116 via the horizontal and vertical slots 150, 152.

The upper variable connection portion 118 of the longitudinal connection plate 116 may include an arcuate slot 156 or a plurality of setting holes (not shown). As such, the rotatable arm 120 may be coupled to the upper variable connection portion 11B via a first fastener 160 at an axis of rotation, and a second fastener 158 extending through the arcuate slot 156 or through at least one of the setting holes.

The front and back vertical support pillars 12, 14 may be plastic and/or metal. The plurality of waist engaging members 32-35 may be formed of plastic, such as cold-moldable or thermosetting plastic, for example.

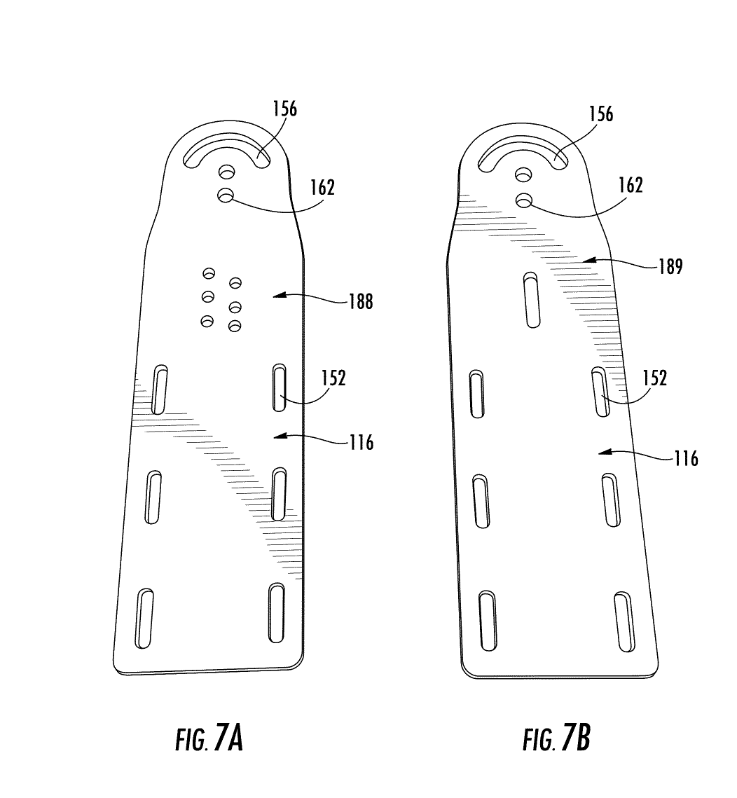

The upper thoracic pad 48 may be coupled between the rotatable arms 20 of the front and back vertical support pillars 12, 14 via at least one strap 72 and associated buckle 74. Likewise, the lower thoracic pad 49 may be coupled between the longitudinal connection plates 16 of the front and back vertical support pillars 12, 14 via at least one strap 82 and associated buckle 84. A fastener 86 may secure the buckle 84 to the longitudinal connection plates 16 at position setting holes 18B or vertical positioning slot 189 (e.g. as illustrated in FIGS. 7A and 7B).

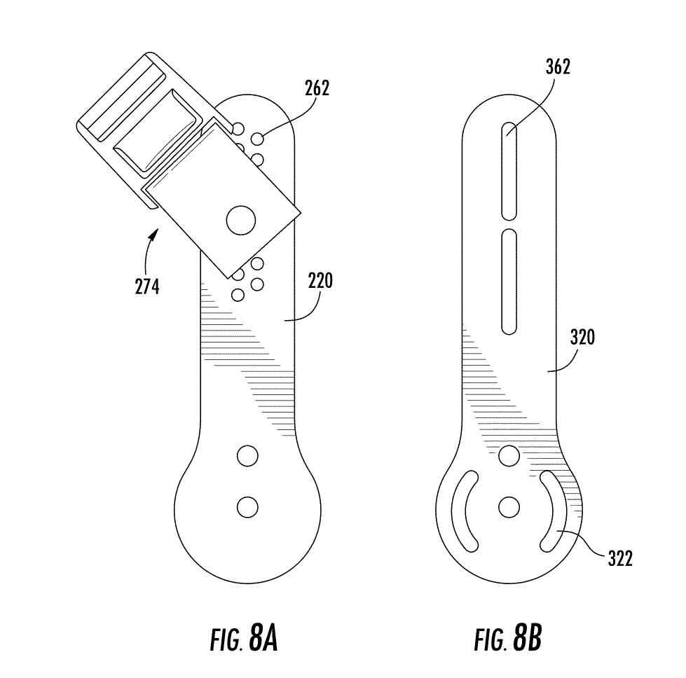

Various embodiments of the rotatable arm 220, 320 are shown in FIGS. 8A and 8B. For example, rotatable arm 220 includes setting holes 262 for attachment of buckle 274, while arm 320 includes slots 362 for attachment of the buckle.

The adjustable scoliosis brace 10 can be fitted and adjusted to fit a patient via the above described features.

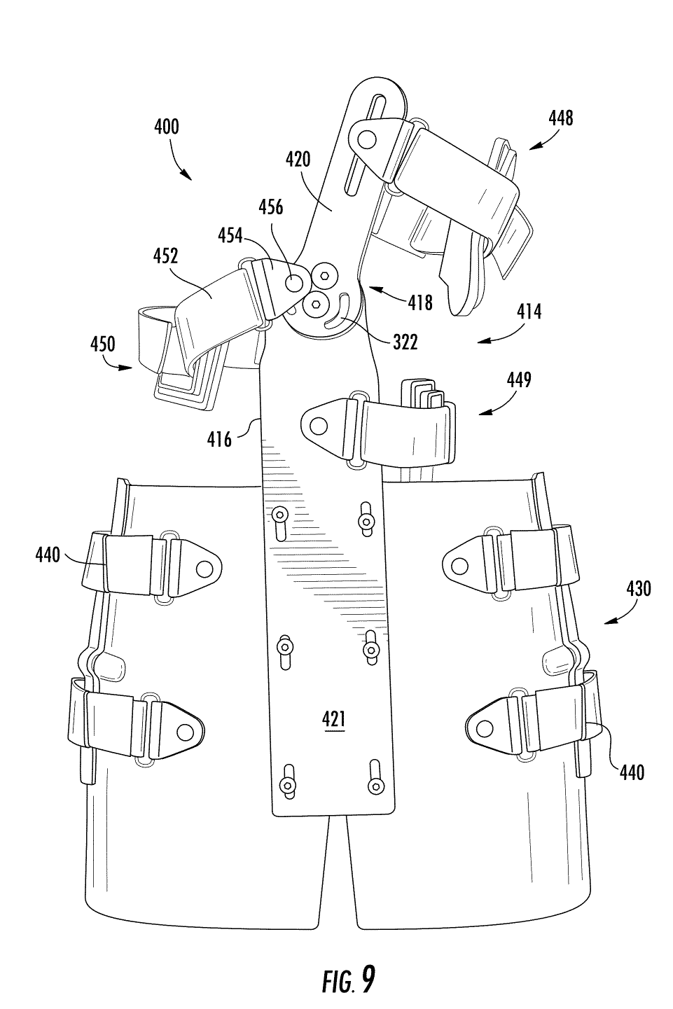

Another embodiment of the adjustable scoliosis brace 400 will be described with reference to FIG. 9. this embodiment of the expandable, adjustable, off-the-shelf brace 400 is effective in treating a spinal curve referred to as a “double curve.” The adjustable scoliosis brace 400 includes front and back vertical support pillars 414 (only the back pillar being shown for convenience), each comprising a longitudinal connection plate 416 including upper and lower variable connection portions 418, 421, and a rotatable arm 420 coupled to the upper variable connection portion of the longitudinal connection plate and extending upwardly therefrom. A girdle device 430 is coupled to the front and back vertical support pillars 414 and configured to surround a waist of the patient.

As in the previous embodiment, the girdle device 430 includes a plurality of waist engaging members contoured to inhibit movement of the brace on a waist of the patient. Again, adjustable coupling features 440, such as straps and buckles, are provided on an external surface of the plurality of waist engaging members and configured to couple the plurality of waist engaging members together. Any number of straps and buckles may be provided to ensure coupling of the waist engaging members.

An upper thoracic pad 448 is configured to be coupled between the rotatable arms 420 of the front and back vertical support pillars 414 and positioned under one shoulder of the patient. A lower thoracic pad 449 is configured to be coupled between the longitudinal connection plates 416 of the front and back vertical support pillars 414 and positioned between the girdle device 430 and the other shoulder of the patient. In this embodiment, a middle thoracic pad 450, including a strap 452 and buckle 454, for example, is configured to be coupled between the upper connection portions 418 of the longitudinal connection plate 416, e.g. via fastener 456 and/or positioning hole 162 (e.g. FIGS. 7A and 7B) and arm slots 322.

The thoracic pads 448, 449 and 450 are adjusted or otherwise set at desired positions and angles, for example, to treat a patient with a double curve, as is understood by those skilled in the art.

It should be understood that in each of the embodiments, the number and/or dimensions of the features such as positioning holes, slots, fasteners, straps, buckles, pads etc. can be varied as needed by the patient and/or desired by the physician.

The primary goal of orthotic treatment for idiopathic scoliosis is to stop the progression of the spinal curve. Bracing may not typically reduce the degree of the curve/amount of angulation already present. Since bracing may only work to stop the progression of the curvature in a growing child, it is not typically used for those children who are already skeletally mature or almost mature. It is mainly used for younger children (e.g. girls who are about 11 to 13 years old, and boys who are about 12 to 14 years).

A method aspect is directed to treating a spinal curve in a patient with an adjustable scoliosis brace 10 the method includes providing front and back vertical support pillars 12, 14 each comprising a longitudinal connection plate 16 including upper and lower variable connection portions 18, 21, and a rotatable arm 20 coupled to the upper variable connection portion of the longitudinal connection plate and extending upwardly therefrom. The method includes coupling a girdle device 30 to the front and back vertical support pillars 12/14 and positioning the girdle device to surround a waist of the patient.

As discussed above, the girdle device 30 includes a plurality of waist engaging members 32-35 contoured to inhibit movement of the brace 10 on a waist of the patient. Adjustable coupling features 40 are provided on an external surface of the plurality of waist engaging members 32-35 and configured to couple the plurality of waist engaging members together. Variable connection features 46 configured to couple the plurality of waist engaging members to the lower variable connection portions 21 of the longitudinal connection plates 16.

The method further includes coupling an upper thoracic pad 48 between the rotatable arms 20 of the front and back vertical support pillars 12/14 and positioning under one shoulder of the patient, and coupling a lower thoracic pad 49 between the longitudinal connection plates 16 of the front and back vertical support pillars 12/14 and positioning between the girdle device 30 and the other shoulder of the patient.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. An adjustable scoliosis brace for treating a spinal curve in a patient and comprising:
front and back vertical support pillars each comprising
a longitudinal connection plate including upper and lower variable connection portions, and
a rotatable arm coupled to the upper variable connection portion of the longitudinal connection plate and extending upwardly therefrom;
a girdle device coupled to the front and back vertical support pillars and configured to surround a waist of the patient, and comprising
a plurality of waist engaging members contoured to inhibit movement of the brace on a waist of the patient,
adjustable coupling features provided on an external surface of the plurality of waist engaging members and configured to couple the plurality of waist engaging members together, and
variable connection features configured to couple the plurality of waist engaging members to the lower variable connection portions of the longitudinal connection plates;
an upper thoracic pad configured to be coupled between the rotatable arms of the front and back vertical support pillars and positioned under one shoulder of the patient; and
a lower thoracic pad configured to be coupled between the longitudinal connection plates of the front and back vertical support pillars and positioned between the girdle device and the other shoulder of the patient.

2. The adjustable scoliosis brace according to claim 1, further comprising padding positioned on interior surfaces of the front and back vertical support pillars, the girdle device and the upper and lower thoracic pads.

3. The adjustable scoliosis brace according to claim 1, wherein the variable connection features of the girdle device comprise horizontal slots adjacent ends of the plurality of waist engaging members.

4. The adjustable scoliosis brace according to claim 3, wherein the lower variable connection portions of the longitudinal connection plates comprise vertical slots; and further comprising fasteners configured to fasten ends of the plurality of waist engaging members to the lower variable connection portions of the longitudinal connection plates via the horizontal and vertical slots.

5. The adjustable scoliosis brace according to claim 1, wherein the upper variable connection portion of the longitudinal connection plate includes an arcuate slot; wherein the rotatable arm is coupled to the upper variable connection portion via a first fastener at an axis of rotation, and a second fastener extending through the arcuate slot.

6. The adjustable scoliosis brace according to claim 1, wherein the upper variable connection portion of the longitudinal connection plate includes a plurality of setting holes; wherein the rotatable arm is coupled to the upper variable connection portion via a first fastener at an axis of rotation, and a second fastener extending through at least one of the setting holes.

7. The adjustable scoliosis brace according to claim 1, wherein the adjustable coupling features provided on the external surface of the plurality of waist engaging members comprise straps and associated buckles.

8. The adjustable scoliosis brace according to claim 1, wherein the front and back vertical support pillars comprise plastic front and back vertical support pillars.

9. The adjustable scoliosis brace according to claim 1, wherein the plurality of waist engaging members comprise plastic waist engaging members.

10. The adjustable scoliosis brace according to claim 1, further comprising at least one strap and associated buckle to couple the upper thoracic pad between the rotatable arms of the front and back vertical support pillars.

11. The adjustable scoliosis brace according to claim 1, further comprising at least one strap and associated buckle to couple the lower thoracic pad between the longitudinal connection plates of the front and back vertical support pillars.

12. An adjustable scoliosis brace for treating a spinal curve in a patient and comprising:
front and back vertical support pillars each comprising
a longitudinal connection plate including upper and lower connection portions, and
a rotatable arm coupled to the upper connection portion of the longitudinal connection plate and extending upwardly therefrom;
a girdle device coupled to the front and back vertical support pillars, and comprising
a plurality of waist engaging members,
coupling features configured to couple the plurality of waist engaging members together at first ends thereof, and
connection features configured to couple the plurality of waist engaging members, at second ends thereof, to the lower connection portions of the longitudinal connection plates;
an upper thoracic pad configured to be coupled between the rotatable arms of the front and back vertical support pillars; and
a lower thoracic pad configured to be coupled between the longitudinal connection plates of the front and back vertical support pillars.

13. The adjustable scoliosis brace according to claim 12, further comprising padding positioned on interior surfaces of the front and back vertical support pillars, the girdle device and the upper and lower thoracic pads.

14. The adjustable scoliosis brace according to claim 12, wherein the connection features of the girdle device comprise first slots adjacent ends of the plurality of waist engaging members.

15. The adjustable scoliosis brace according to claim 14, wherein the lower variable connection portions of the longitudinal connection plates comprise second slots; and further comprising fasteners configured to fasten ends of the plurality of waist engaging members to the lower connection portions of the longitudinal connection plates via the first and second slots.

16. The adjustable scoliosis brace according to claim 12, wherein the upper connection portion of the longitudinal connection plate includes an arcuate slot; wherein the rotatable arm is coupled to the upper connection portion via a first fastener at an axis of rotation, and a second fastener extending through the arcuate slot.

17. The adjustable scoliosis brace according to claim 12, wherein the upper connection portion of the longitudinal connection plate includes a plurality of setting holes; wherein the rotatable arm is coupled to the upper connection portion via a first fastener at an axis of rotation, and a second fastener extending through at least one of the setting holes.

18. The adjustable scoliosis brace according to claim 12, wherein the front and back vertical support pillars comprise plastic front and back vertical support pillars.

19. The adjustable scoliosis brace according to claim 12, wherein the plurality of waist engaging members comprise plastic waist engaging members.

20. A method of treating a spinal curve in a patient with an adjustable scoliosis brace, the method comprising:
providing front and back vertical support pillars each comprising
a longitudinal connection plate including upper and lower variable connection portions, and
a rotatable arm coupled to the upper variable connection portion of the longitudinal connection plate and extending upwardly therefrom;
coupling a girdle device to the front and back vertical support pillars and positioning the girdle device to surround a waist of the patient, and the girdle device comprising
a plurality of waist engaging members contoured to inhibit movement of the brace on a waist of the patient,
adjustable coupling features provided on an external surface of the plurality of waist engaging members and configured to couple the plurality of waist engaging members together, and
variable connection features configured to couple the plurality of waist engaging members to the lower variable connection portions of the longitudinal connection plates;
coupling an upper thoracic pad between the rotatable arms of the front and back vertical support pillars and positioning under one shoulder of the patient; and
coupling a lower thoracic pad between the longitudinal connection plates of the front and back vertical support pillars and positioning between the girdle device and the other shoulder of the patient.

21. The method according to claim 20, further comprising positioning padding on interior surfaces of the front and back vertical support pillars, the girdle device and the upper and lower thoracic pads.

22. The method according to claim 20, wherein the variable connection features of the girdle device comprise horizontal slots adjacent ends of the plurality of waist engaging members.

23. The method according to claim 22, wherein the lower variable connection portions of the longitudinal connection plates comprise vertical slots; and further comprising fastening ends of the plurality of waist engaging members to the lower variable connection portions of the longitudinal connection plates via fasteners through the horizontal and vertical slots.

24. The method according to claim 20, wherein the upper variable connection portion of the longitudinal connection plate includes an arcuate slot; wherein the rotatable arm is coupled to the upper variable connection portion via a first fastener at an axis of rotation, and a second fastener extending through the arcuate slot.

25. The method according to claim 20, wherein the upper variable connection portion of the longitudinal connection plate includes a plurality of setting holes; wherein the rotatable arm is coupled to the upper variable connection portion via a first fastener at an axis of rotation, and a second fastener extending through at least one of the setting holes.

26. The method according to claim 20, wherein the adjustable coupling features provided on the external surface of the plurality of waist engaging members comprise straps and associated buckles.

27. The method according to claim 20, wherein the front and back vertical support pillars comprise plastic front and back vertical support pillars.

28. The method according to claim 20, wherein the plurality of waist engaging members comprise plastic waist engaging members.
